(19) United States
(12) Patent Application Publication
(10) Pub. No.: US 2026/0020932 A1
(43) Pub. Date: Jan. 22, 2026

(54) Glove Dispenser Soft Pack with Elastic Band Strap

(71) Applicant: O&M Halyard, Inc., Mechanicsville, VA (US)

(72) Inventors: Chelsey E. McClain, Atlanta, GA (US); Shantilal H. Modha, Milton, GA (US); Frank J. Walker, III, Duluth, GA (US); Marshall R. Dean, Cumming, GA (US); Khoa T. Lien, Alpharetta, GA (US); Patrick H. Robert, Alpharetta, GA (US)

(21) Appl. No.: 18/994,722

(22) PCT Filed: Jul. 20, 2023
(86) PCT No.: PCT/US2023/028242

§ 371 Date: Jan. 15, 2025

Related U.S. Application Data
(60) Provisional application No. 63/390,996, filed on Jul. 21, 2022.
(60) Provisional application No. 63/390,998, filed on Jul. 21, 2022.
(60) Provisional application No. 63/433,036, filed on Dec. 16, 2022.

(51) Int. Cl. A61B 42/40; B65D 21/08; B65D 25/22; B65D 83/08

(52) U.S. Cl. CPC A61B 42/40; B65D 21/08; B65D 25/22; B65D 83/0805; B65D 83/0894

(57) ABSTRACT

A collapsible dispenser includes a dispenser body including a top, a bottom, and at least one side defining an enclosed space having a volume, and a resealable opening into the enclosed space. The collapsible dispenser may further include an elastic strap coupled to the dispenser body. The dispenser is configured to collapse as contents contained within the dispenser are dispensed. A glove dispensing assembly further includes a collapsible dispenser surrounding a stack of gloves, the dispenser defining an opening through which the gloves are dispensed.

### RELATED APPLICATIONS

The present application claims priority to U.S. Provisional Application Ser. No. 63/390,996, filed on Jul. 21, 2022, U.S. Provisional application Ser. No. 16/390,998, filed on Jul. 21, 2022, and U.S. Provisional Application Ser. No. 63/433,036, filed on Dec. 16, 2022, which are incorporated herein in their entirety by reference thereto.

### FIELD OF THE INVENTION

The subject matter of the present invention relates generally to glove packaging and dispensing systems.

### BACKGROUND

Elastic gloves made from natural latex polymers or from synthetic polymers are used in numerous applications. Such gloves, for instance, are used by medical personnel during examinations, surgeries, and the like. Elastic gloves are also used during food preparation and in clean room environments. The gloves are used to protect the hands of a wearer and/or to prevent contamination. Elastic gloves may sometimes be desirable to be worn by individuals in situations where contact with certain substances is undesirable, for example when using chemicals for cleaning or when changing a diaper.

The elastic gloves can be made from numerous materials. For instance, as described above, in one embodiment, the gloves are made from natural latex polymers. Synthetic polymers that are used to make elastic gloves include polyvinyl chloride, nitrile polymers, polychloroprene, polyisoprene, block copolymers such as styrene-ethylene butylene-styrene block copolymers, and the like.

Elastic gloves are commonly packaged together in dispensers that are intended to dispense the gloves one at a time. Such dispensers are very useful especially in environments where new gloves are constantly needed. For instance, physicians or nurses performing medical examinations and tests are constantly discarding old gloves and donning new gloves as they see new patients.

Furthermore, dispenser packages for elastic gloves are typically inconvenient, particularly for on-the-go use. One type of typical dispenser package is a rigid container made of either metal, plastic, or cardboard, which is difficult to carry around because it takes up a large amount of space and is inflexible. These rigid containers often have sharp edges, so they cannot easily be carried in a purse, diaper bag, medical kit, toolbox, automobile glove compartment, or other portable carrier. As gloves are removed from rigid containers, remaining gloves rest at the bottom of the container, so wearers need to dig deeper and deeper within the container to reach the remaining gloves. This greatly increases the risk of cross-contamination because multiple gloves may be touched with every reach. Additionally, as the containers become less full there is less predictability of what area of the gloves the user may be touching or pulling, and gloves may be pulled out by the palm or finger areas of the glove. This may potentially contaminate the finger and palm areas of the glove, which need to stay clean. Furthermore, reaching deep into the container to dispense a glove may result in involuntarily dispensing multiple gloves from the container at once, which often results in contamination, by putting the unnecessary gloves back into the container, or waste, by discarding the unnecessary gloves.

An alternative existing dispenser for elastic gloves is a soft dispenser in the form on a plastic wrap with gloves stuffed inside. However, gloves are often stuffed inside these dispensers without careful organization or packaging. There can be a large amount of friction generated between the tacky external surface of each of the gloves against the plastic wrap. As a result, gloves often stick together and stick to the plastic packaging, making it difficult to avoid multiple dispensing and cross-contamination of the gloves. Further, existing soft plastic dispensers do not have an easy opening and are not re-sealable. As a result, once the dispenser has been opened, gloves may easily come out of the dispenser unintentionally, and debris from outside the dispenser may get inside the unsealed opening, potentially contaminating the remaining gloves in the dispenser.

Consequently, there is a need for an improved glove dispenser assembly that addresses the aforementioned problems. In particular, a glove dispenser that is easily portable and collapsible would also be useful.

### SUMMARY

Aspects and advantages of the invention in accordance with the present disclosure will be set forth in part in the following description, or may be obvious from the description, or may be learned through practice of the technology.

In one aspect, the present invention is directed to a collapsible dispenser. The dispenser includes a dispenser body including a top, a bottom, and at least one side defining an enclosed space having a volume, wherein the dispenser body is formed from a flexible material; and an opening into the enclosed space being located on the top, the bottom, or a side of the at least one side of the dispenser body. The dispenser further includes an elastic strap coupled to the dispenser body. The dispenser is configured to collapse as contents contained within the dispenser are dispensed.

In another aspect, the dispenser may further include a rigid lid, wherein the rigid lid comprises a rigid perimeter coupled to the top, bottom, or side of the at least one side of the dispenser body and a movable lid coupled to the rigid perimeter, the rigid perimeter including at least one retaining element configured to retain the elastic strap, and the elastic strap being coupled to the rigid perimeter. Further, the at least one retaining element may include a tab extending from an edge of the rigid perimeter. Moreover, the at least one retaining element may include a pair of retaining elements disposed on opposing first and second sides of the rigid perimeter. Further, the at least one retaining element may further include at least one indentation in a surface of the rigid perimeter upon which the elastic strap is configured to rest. Moreover, the at least one retaining element may include at least one slot extending through the rigid perimeter, each slot being formed between an external edge of the rigid perimeter and an internal edge of the rigid perimeter, the at least one slot being configured to receive an elastic band or an external strap or hook.

In a further aspect, the elastic strap may include an elongated body extending from a first end to a second end, further comprising a strap coupling element at each of the first end and the second end.

In still another aspect, the elastic strap may have an elongated position and a relaxed position, further wherein the elastic strap is coupled to the dispenser body in the elongated position and, as contents of the dispenser are dispensed, the elastic strap may be configured to move into the relaxed position, wherein as contents of the dispenser are dispensed and the elastic strap moves into the relaxed position, the dispenser body is configured to shrink in size and the volume of the enclosed space is configured to be reduced as the contents of the dispenser are dispensed.

In one more aspect, the elastic strap may have an elastic ring configured to surround the dispenser body, wherein the elastic ring may be configured to cover the opening. Further, the elastic ring may include a slit configured to align with the opening of the dispenser body to permit contents of the dispenser to be dispensed through the slit.

The present invention is further directed to a glove dispensing assembly. The assembly includes a collapsible dispenser and a stack of interfolded gloves. The dispenser includes a dispenser body including a top, a bottom, and at least one side defining an enclosed space having a volume, the top, bottom, and at least one side each being formed from a flexible material; and a resealable opening into the enclosed space located on the top, the bottom, or a side of the at least one side. In the stack of interfolded gloves, each glove in the stack including a finger end and a cuff end, the stack including an initial glove and a plurality of subsequent gloves. The stack of interfolded gloves being disposed in the enclosed space of the dispenser. The dispenser is configured to collapse as gloves of the stack of gloves contained within the enclosed space are dispensed such that the volume of the enclosed space decreases.

In one aspect of the assembly, the dispenser may further include a lid sealing the resealable opening. Further, an inner surface of the lid may further include an adhesive region. Moreover, the lid may include single piece of rigid material comprising an outer perimeter coupled to the flexible material and a movable member configured to move between an open configuration in which the opening is exposed and a closed configuration in which the opening is covered, a movable portion of the lid being a cut-out portion of the single piece of rigid material. Further, at least a portion of the flexible material may be exposed within the outer perimeter of the lid when the movable member is in the open configuration. Moreover, the movable portion may have a cantilever snap and the outer perimeter having a corresponding slot in which the cantilever snap is configured to lock in place in the closed configuration.

In another aspect, the assembly may further include an elastic strap coupled to the dispenser body; the elastic strap being configured to compress the collapsible dispenser.

In a further aspect, the assembly may further include a mounting element coupled to the dispenser body, wherein the mounting element is configured to mount the dispenser to a surface. For instance, the mounting element may include an adhesive region comprising an adhesive material configured to adhere to the surface. Moreover, the dispenser may include an elongated section comprising the mounting element, and the mounting element comprises one or more openings configured to receive a belt or other accessory therethrough.

These and other features, aspects, and advantages of the present invention will become better understood with reference to the following description and appended claims. The accompanying drawings, which are incorporated and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 illustrates a perspective view of a glove dispenser assembly of the present invention;

FIG. 2 illustrates a top view of an exemplary elastic band and dispenser lid of the dispenser assembly of FIG. 1;

FIG. 3A illustrates a top view of a dispenser lid of one embodiment of the present invention in an open condition;

FIG. 3B illustrates a top view of the dispenser lid of FIG. 3A in a closed condition;

FIG. 4A illustrates a top view of a dispenser lid according to another embodiment of the present invention in an open condition;

FIG. 4B illustrates a top view of the dispenser lid of FIG. 4A in a closed condition;

FIG. 5A illustrates a top view of a dispenser lid according to another embodiment of the present invention in an open condition;

FIG. 5B illustrates a top view of the dispenser lid of FIG. 5A in a closed condition;

FIG. 6A illustrates a top view of a dispenser lid according to another embodiment of the present invention in an open condition;

FIG. 6B illustrates a top view of the dispenser lid of FIG. 6A in a closed condition;

FIG. 7A illustrates a top view of a dispenser lid according to another embodiment of the present invention in an open condition;

FIG. 7B illustrates a top view of the dispenser lid of FIG. 7A in a closed condition;

FIG. 8A illustrates a top view of a dispenser lid according to another embodiment of the present invention in an open condition;

FIG. 8B illustrates a top view of the dispenser lid of FIG. 8A in a closed condition;

FIG. 9A illustrates a top view of a dispenser lid according to another embodiment of the present invention in an open condition;

FIG. 9B illustrates a top view of the dispenser lid of FIG. 9A in a closed condition;

FIG. 10A illustrates a top view of a dispenser lid according to another embodiment of the present invention in an open condition;

FIG. 10B illustrates a top view of the dispenser lid of FIG. 10A in a closed condition;

FIG. 11 illustrates a top view of a dispenser lid according to another embodiment of the present invention in an open condition;

FIG. 12 illustrates a perspective view of a glove dispenser assembly according to another embodiment of the present invention;

FIG. 13 illustrates a perspective view of a glove dispenser assembly according to another embodiment of the present invention;

FIG. 14A illustrates an assembly of the glove dispenser assembly of FIG. 13;

FIG. 14B illustrates the glove dispenser assembly of FIG. 13 being compressed by an elastic strap;

FIG. 15 illustrates a perspective view of a glove dispenser assembly according to another embodiment of the present invention; and

FIG. 16 illustrates the glove dispenser assembly of FIG. 15 in use.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents.

As used herein, the terms “first”, “second”, and “third” may be used interchangeably to distinguish one component from another and are not intended to signify location or importance of the individual components. The singular forms “a,” “an,” and “the” include plural references unless the context clearly dictates otherwise. The terms “coupled,” “fixed,” “attached to,” and the like refer to both direct coupling, fixing, or attaching, as well as indirect coupling, fixing, or attaching through one or more intermediate components or features, unless otherwise specified herein. As used herein, the terms “comprises,” “comprising,” “includes,” “including,” “has,” “having” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Terms of approximation, such as “about,” “generally.” “approximately,” or “substantially,” include values within ten percent greater or less than the stated value. When used in the context of an angle or direction, such terms include within ten degrees greater or less than the stated angle or direction. For example, “generally vertical” includes directions within ten degrees of vertical in any direction, e.g., clockwise or counter-clockwise.

Benefits, other advantages, and solutions to problems are described below with regard to specific embodiments. However, the benefits, advantages, solutions to problems, and any feature(s) that may cause any benefit, advantage, or solution to occur or become more pronounced are not to be construed as a critical, required, or essential feature of any or all the claims.

Generally speaking, the present invention is directed to a collapsible dispenser and a glove dispensing assembly including a collapsible dispenser. The dispenser includes a dispenser body defining an enclosed space having a volume, and a resealable opening into the enclosed space. The dispenser further includes an elastic strap coupled to the dispenser body. The dispenser is configured to collapse as contents contained within the dispenser are dispensed. A glove dispensing assembly including a collapsible dispenser enclosing a stack of gloves is also provided. The present inventors have found that the collapsible dispenser and glove dispensing assembly of the present disclosure have improved collapsibility resulting from the compression of the elastic strap against the dispenser body, thereby reducing the volume of the dispenser and improving the ease of transport of the dispenser and glove dispensing assembly.

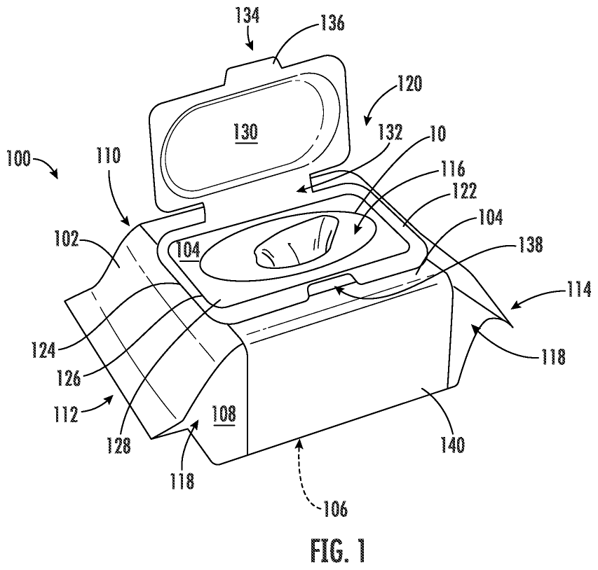

The specific features of the glove dispenser assembly of the present invention may be better understood with reference to FIGS. 1-18C.

The gloves that are contained in the glove dispensing assembly of the present disclosure can vary depending upon the particular application. The gloves can be made from any sheet-like material. The gloves, for instance, can be made from a woven fabric, a knitted fabric, or a nonwoven fabric. The gloves can also be made from a fabric laminate. In one particular embodiment, the gloves are made from an elastic material, such as an elastic polymer. For instance, the gloves can be made from a natural rubber latex polymer or from a synthetic polymer. Synthetic polymers that are used to produce gloves include polyvinyl chloride and other vinyl plastisols, neoprene, nitrile rubber, and the like. The elastic gloves can also be formed from a styrenic polymer and/or from a block copolymer. For instance, the gloves can be formed from a styrene-butylene-styrene polymer or a styrene-ethylene-butylene-styrene polymer. In yet another embodiment, the gloves can be made from a styrene-isoprene-styrene polymer.

The gloves contained in the dispensing assembly can be interfolded. For example, the interfolded gloves may include at least two folds in an S-like configuration. The at least one additional fold compared to existing glove dispensing assemblies that use a single-fold C-like configuration enables the gloves contained within the glove dispensing assembly of the present invention to be dispensed in a lateral direction to the glove fold or in a perpendicular direction to the glove fold. The at least double fold design also prevents against gloves being unintentionally removed from the stack. In particular, the at least double fold design prevents against dispensing gloves from the stack that are not pulled upon at the cuff of the glove by a user for dispensing.

Referring now to FIG. 1, one embodiment of glove dispenser assembly 100 of the present invention is shown. The glove dispenser assembly 100 includes a dispenser body 102 containing a stack of gloves 10. The stack of gloves 10 includes an initial glove and a plurality of subsequent gloves. The number of gloves in any given assembly may vary depending on various factors. In general, the stack of gloves 10 of the glove dispensing assembly 100 can contain from about 5 to about 500 gloves, such as from about 50 gloves to about 100 gloves.

The dispenser 100 can be a resealable dispenser 100 formed from soft, flexible materials. For example, the dispenser 100 may be made from plastic, a biodegradable material, an elastomeric material, or any other suitable soft, flexible materials. In one aspect of the present invention, the dispenser 100, e.g., the dispenser body 102, may be formed from polyethylene; however, the dispenser 100 may also be formed from polypropylene, other polymers, or combination(s) thereof. The dispenser body 102 includes at least a top surface 104, a bottom surface 106, a front surface 108, and a back surface 110. In the embodiment illustrated in FIG. 1. the dispenser body 102 additionally includes a first edge 112 and a second edge 114 extending from the front surface 108 to the back surface 110 to form an envelope or pouch-style dispenser. Side gussets 118 are formed between the first edge 112 and the front surface 110, the second edge 114 and the front surface 108, the first edge 112 and the back surface 110, and the second edge 114 and the back surface 110, respectively. The top surface 104, bottom surface 106, front surface 108, back surface 110, first edge 112 and second edge 114 define an enclosed space having a volume. The dispenser body 102 includes an opening 116 into the enclosed space. The opening 116 can be formed through the top surface 104, as shown in FIG. 1, or in any of the front surface 108, bottom surface 106, or rear surface 110.

Although FIG. 1 illustrates a dispenser body 102 having an envelope or pouch-style dispenser shape, the dispenser body 102 may be formed in any other suitable shape, such as a rectangular prism shape having generally perpendicular sides.

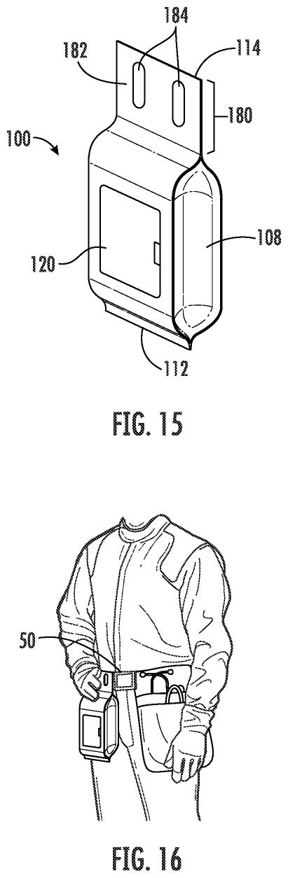

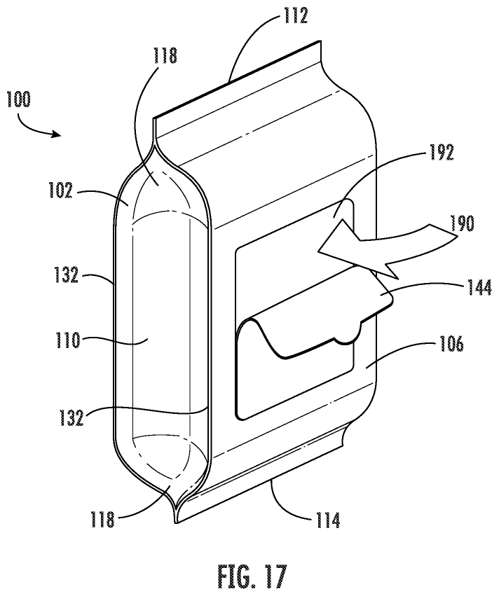

For instance, as shown in FIGS. 15-16, either or both of the first edge 112 and the second edge 114 can have an elongated section 180 formed along with the front surface 108 and back surface 110 to form a coupling member 182. The coupling member 182 can include, e.g., one or more openings 184 configured to receive a belt or other accessory 50 therethrough such that the elongated section 180 may form a belt loop or other coupler to enable easy carrying for a user.

As illustrated in FIGS. 17 and 18A-18C, the glove dispensing assembly 100 can include a mounting element 190 coupled to at least one surface of the dispenser body 102. For example, when the opening 116 is formed between the top 104 and front 110 surfaces of the dispenser body 102, the mounting element 190 may be advantageously disposed on the bottom 106, as shown in FIG. 17, or back 110 surfaces of the dispenser body 102 to provide ease of access to the opening 116 for dispensing.

As shown in FIG. 17, the mounting element 190 may be formed from an adhesive region 192 on at least one surface of the dispenser body 102. The mounting element 190 can further include at least one protective cover sheet 194 over the adhesive region 192 that is configured to protect the adhesive until the glove dispensing assembly 100 is ready for mounting. Then, the protective cover sheet 194 can be peeled away from the adhesive region 192 to expose an adhesive material, such as a pressure sensitive adhesive, that is configured to adhere to a surface. The adhesive material can be any suitable adhesive configured to hold the dispenser body 102 in place during dispensing of one or more gloves through the opening 116 while being removably adhered so the dispenser body 102 can be removed from the surface, e.g., for disposal.

FIG. 18A illustrates the glove dispensing assembly 100 mounted to a vertical wall 200a. FIG. 18B illustrates the glove dispensing assembly 100 mounted to a horizontal surface 200b, e.g., a table top or workspace. FIG. 18C illustrates the glove dispensing assembly undermounted to a horizontal surface 200b, e.g., mounted beneath a table or work surface. In each mounting configuration, the gloves can be dispensed through the opening 116 of the dispenser body 102 and the opening 116 of the lid 120 in a direction that is horizontal or vertical with respect to the surface upon which the assembly 100 is mounted as a result of the opening being disposed along an edge of the dispenser body 102. By enabling both horizontal and vertical mounting and dispensing, and by holding the assembly 100 in place with the mounting element 190, e.g., adhesive region 192, to provide a counter-force when a user is removing a glove, the present inventors have found that the ease of dispensing the gloves, particularly when using only one hand, has been significantly improved.

The opening 116 into the enclosed space of the dispenser body 102 may optionally be sealed by a lid 120 as shown in FIG. 1. For instance, the lid 120 may be formed as a rigid lid. In other words, whereas the dispenser body 102 may be formed from a flexible and/or soft material, as described above, the lid 120 can be formed from a rigid material with little to no flexibility. The rigid lid 120 can be coupled to the dispenser body 102 on the same surface as the opening 116 into the enclosed space of the dispenser body 102. As illustrated in FIG. 1, the opening 116 can be formed in the top surface 104 of the dispenser body 102 and the rigid lid 120 can be coupled to the top surface 104 and surrounding the opening 116. The rigid lid 120 can be coupled to the dispenser body 102 by an adhesive, such as a permanent adhesive, or any other suitable attachment means.

The rigid lid 120 may have a rigid perimeter 122 and a movable lid 130 that is fixed to the perimeter 122 by at least one movable hinge 132. The rigid perimeter 122 has an outer perimeter edge 124 and an inner perimeter edge 126, and an opening 128 defined by the inner perimeter edge 126. When the movable lid 130 is in an open configuration, as shown in FIG. 1, the opening 116 into the enclosed space of the dispenser body 102 can be accessed through the opening 128 of the rigid perimeter 122.

In one particular embodiment, the rigid lid 120 is formed from a single piece of material and the movable lid 130 is a cut-out portion of the single piece of material that is attached at a flexible joint 132 that allows the movable lid 130 to fold open and close. In another embodiment (not shown), the movable lid 130 may be formed from a separate piece of material and attached to the perimeter 122 at a hinge joint 132, such as a barrel hinge, a spring hinge, pivot hinge, or other type of hinge. The rigid lid 120 may have a locking mechanism 134 to lock the movable lid in place to seal the opening 116. For example, the locking mechanism may be a snap, wherein the movable lid 130 includes at least one cantilever snap having a locking surface 136, and the rigid outer perimeter 124 includes at least one corresponding slot 138 in which the locking surface 136 may lock in place.

Additionally or alternatively, the opening 116 may be sealed by a sticker or peelable sheet adhered to the top surface 104 of dispenser body 102 by adhesive on the top surface 104. The sticker can be peeled open at the opening 116 to dispense the contents, e.g. glove, of the dispenser, and then stuck back over the opening 116 to the adhesive to seal the opening 116. For example, a peelable sheet can be adhered to the top surface 104 within the inner perimeter edge 126 of the rigid lid 120. The peelable sheet can be disposable or reusable. In other words, the peelable sheet can be configured to be a single-use seal that is disposed of after the first time the peelable sheet is removed from the top surface 104, or the peelable sheet can be configured to be resealed over the opening 116 against the top surface 104.

The dispenser 100 is a collapsible dispenser. For example, a collapsible dispenser body 102 may be formed from flexible materials such that the dispenser 100 shrinks in size as its contents (e.g., gloves) are dispensed and removed from the dispenser. For example, the dispenser body 102 can be formed from one or more elastomeric materials. In this embodiment, the elastomeric material of the dispenser 100 can be stretched to fit a plurality of contents (e.g., gloves) within the dispenser in a flexible manner. As the gloves are removed from the dispenser 100, the elastomeric material shrinks, reducing the enclosed space of the dispenser, until it returns to its unstressed shape, enabling the dispenser 100 to take up minimum space at all times. Because the elastomeric material is soft and flexible, when the elastomeric material reaches its unstressed state, the dispenser remains soft and flexible and is thereby easy and convenient to carry in a purse, diaper bag, medical kit, tool box, or an otherwise portable fashion. A collapsible dispenser 100 may also be formed from soft, flexible plastic or other materials that are not elastomeric. For example, a flexible plastic dispenser may be able to be compressed as contents of the dispenser are dispensed to reduce the volume of air in the enclosed space. In one embodiment, the collapsible dispenser may be formed of shrink-wrapped material.

Additionally or alternatively, as shown in FIG. 1 and described in further detail below, the collapsible dispenser 100 can include one or more elastic straps 140 coupled to the dispenser body 102 that are configured to compress the dispenser body 102 to collapse as the contents of the dispenser are dispensed.

The elastic strap 140 includes a strap body 142 formed from an elastic material. For instance, the material can include natural or synthetic rubber, isoprene, neoprene, or any combination thereof. The elastic strap 140 can be coupled to the dispenser body 102 and/or the rigid lid 120. For instance, as shown in FIG. 1, the elastic strap 140 may be coupled between the dispenser body 102 and the rigid lid 120, e.g., by an adhesive, permanent bonding, or any other suitable attachment means.

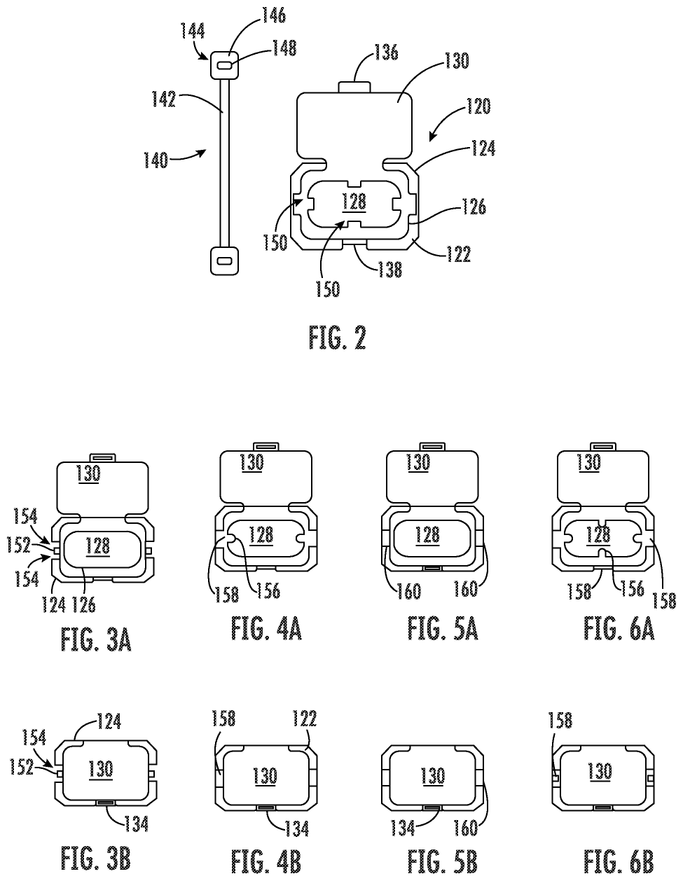

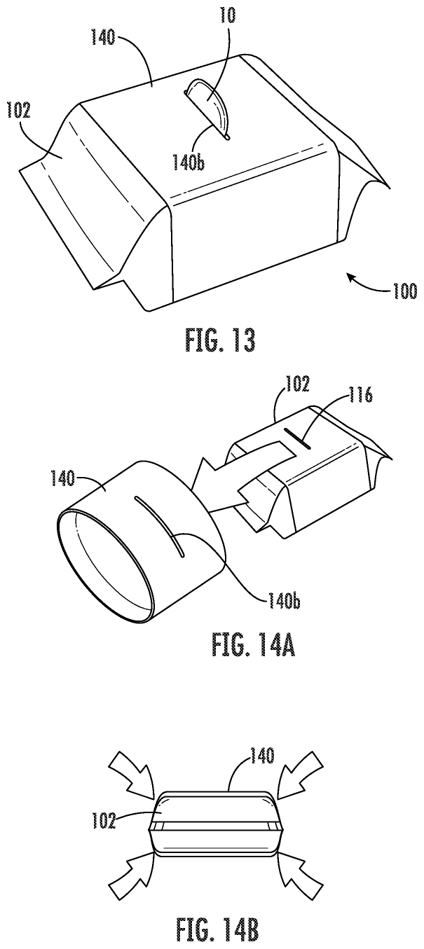

FIG. 2 illustrates an aspect of the elastic strap according to the present invention. The elastic strap 140 can have one or more strap coupling elements 144 coupled to the strap body 142. As shown in FIG. 2, the strap body 142 may be an elongate strap body having first and second strap coupling elements 144 at respective ends of the elongate strap body 142. The strap coupling elements 144 can have a coupling body 146 and one or more slots 148 configured to couple with a cooperating retaining element 150. As shown in FIG. 2, the rigid lid 120 of the dispenser 100 can include one or more retaining elements 150 configured to cooperatively receive a strap coupling element 144 to couple the elastic strap 140 to the rigid lid 120. In another aspect of the present invention, the elastic strap 140 can have a continuous loop or circular configuration, e.g., as illustrated in FIGS. 13-14B and described in further detail below, rather than an elongated shape. The continuous loop may optionally be provided with one or more strap coupling elements 144.

Each of the FIGS. 3A-10A and 3B-10B illustrate exemplary arrangements of rigid lids 120 having one or more retaining elements 150 to receive and couple the elastic strap 140. As illustrated, FIGS. 3A-10A each illustrates aspects of a rigid lid 120 with the movable lid 130 in an open configuration, and FIGS. 3B-10B each illustrates aspects of a rigid lid 120 with the movable lid 130 in a closed configuration.

Turning to FIGS. 3A and 3B, the retaining element(s) 150 can include one or more external tabs 152 extending from the outer perimeter edge 124 of the rigid perimeter 122. As shown, the outer perimeter edge 124 can have one or more external indentations 154 extending in a direction toward the inner perimeter edge 126. The external tabs 152 can extend from within or between external indentations 154. A slot 148 of a strap coupling element 144 can receive an external tab 152 inserted therein in order to retain the strap coupling element 144 in place with respect to the lid 120. In another aspect, an elastic strap 140 having a loop configuration may be inserted within the indentations 154 and over the external tabs 152 to retain the looped elastic band 140 in place. As shown in FIGS. 3A-B, the rigid perimeter 122 can include two external tabs 152 extending from opposing sides of the rigid perimeter 122, e.g., right and left lateral sides of the rigid perimeter 122.

Referring now to FIGS. 4A-B, the retaining element(s) 150 can include one or more internal tabs 156 extending from the inner perimeter edge 126 of the rigid perimeter 122. Additionally, an upper surface of the rigid perimeter 122 can include an indented surface 158 extending from the outer perimeter edge 124 to the inner perimeter edge 126. The indented surface 158 can be configured to receive a portion of the strap body 142 of the elastic strap 140 such that the strap body 142 can extend across the rigid perimeter 122 and the strap coupling element 144 can be coupled to the internal tab 156. As shown in FIGS. 4A-B, the rigid perimeter 122 can include two internal tabs 156 extending from opposing sides of the rigid perimeter 122, e.g., right and left lateral sides of the rigid perimeter 122.

Turning now to FIGS. 5A-B, 7A-B, 8A-B, 9A-B and 10A-B, the retaining element(s) 150 can include one or more slots 160 extending between the outer perimeter edge 124 and the inner perimeter edge 126 of the rigid perimeter 122. The slot(s) 160 can be configured to receive a strap body 142 of an elastic strap 140 and/or a strap coupling element 144 of an elastic strap 140. Additionally or alternatively, the slot(s) 160 can be configured to receive an external coupling element, e.g., a strap, hook, carabiner, belt, or any other suitable coupling element configured to couple to and retain the dispenser 100.

For instance, as shown in FIGS. 5A-B, the slots 160 can be formed as generally elongated, e.g., rectangular, slots 160. The elongated or rectangular slots 160 can be configured to receive an elastic strap body 142.

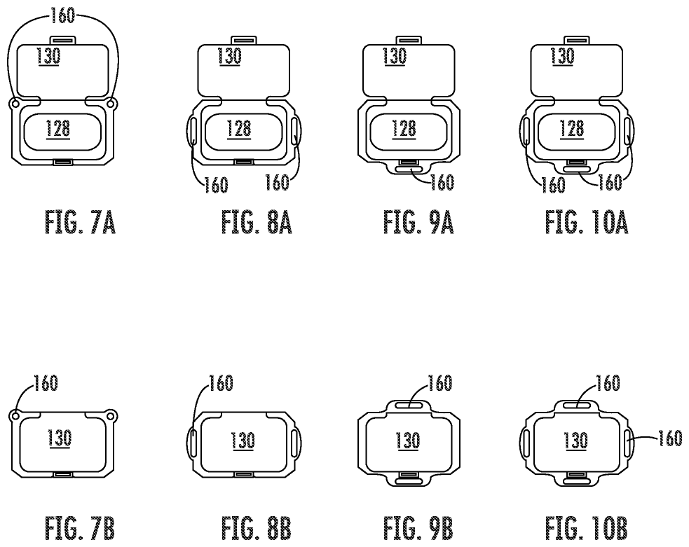

As illustrated in FIGS. 7A-B, the slots 160 can be formed as circular openings 160. Additionally, FIGS. 7A-B illustrate an arrangement contemplated by the present disclosure in which the retaining elements 150 are disposed along a same side of the perimeter 122, e.g., in adjacent corners of the perimeter 122 as shown in FIGS. 7A-B.

FIGS. 8A-B, 9A-B and 10A-B illustrate slots 160 having a generally oval shape. As shown in FIGS. 8A-B and FIGS. 9A-B, the rigid perimeter 122 can include two slots 160 on opposing sides of the rigid perimeter 122, e.g., right and left lateral sides of the rigid perimeter 122 as shown in FIGS. 8A-B or top and bottom sides of the rigid perimeter as shown in FIGS. 9A-B. The present inventors have found that the oval shaped slots 160 can provide a versatile attachment point that may be suitable for both the elastic strap coupling body 146 and for external coupling element, e.g., a strap, hook, carabiner, belt, or any other suitable coupling element configured to couple to and retain the dispenser 100.

In some aspects of the present disclosure, and as best illustrated in FIG. 6A and FIG. 10A-B, the retaining element(s) 150 can include multiple pairs of retaining elements 150. Each pair of retaining elements 150 can optionally be disposed on opposing surfaces of the rigid perimeter 122. In such an arrangement, the dispenser 100, via the rigid lid 120, may be optionally coupled and/or mounted in a lateral direction or a longitudinal direction. For instance, FIG. 6A illustrates a first pair of internal tabs 156 on right and left lateral sides of the rigid perimeter 122, and a second pair of internal tabs 156 on top and bottom sides of the rigid perimeter. FIGS. 10A-B illustrate a similar arrangement with slots 160, in which there is a first pair of slots 160 on right and left lateral sides of the rigid perimeter 122, and a second pair of slots on top and bottom sides of the rigid perimeter.

The collapsible dispenser 100 can be configured to contain a stack of gloves 10 as illustrated in FIG. 1. The stack of gloves 10 can be folded in an S-fold configuration having two folds per glove. Additionally or alternatively, the collapsible dispenser 100 can be configured to contain a stack of gloves having a single fold (e.g., a C-shaped fold), a stack of gloves having no folds, a plurality of gloves that are not stacked or arranged in any particular order, or the collapsible dispenser 100 can have any other configuration in which the collapsible dispenser 100 contains a plurality of contents. For example, the collapsible dispenser 100 can be configured to contain a stack of gloves having pairs of 2 gloves folded together with a single fold so as to dispense two gloves simultaneously.

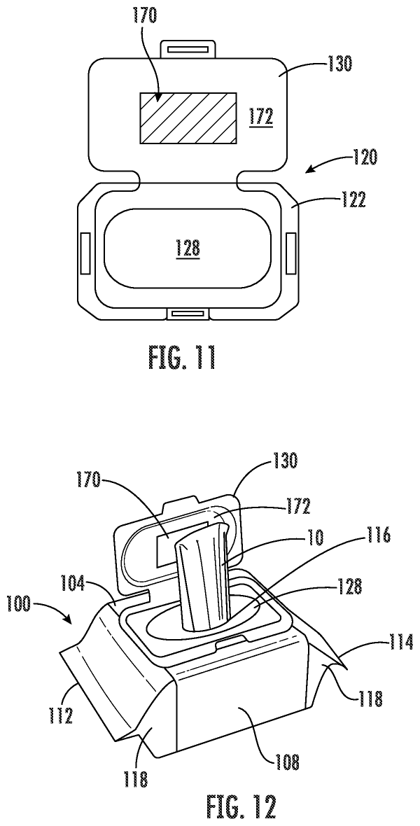

Turning to FIGS. 11 and 12, the dispenser 100 can further include an adhesive region 170 configured to aid with dispensing the gloves 10. The adhesive region 170 can be formed on an inner surface 172 of the movable lid 130 that faces the opening 116 into the enclosed space of the dispenser body 102. As such, the adhesive region 170 can contact a first glove 10 in the stack when the movable lid 130 is in a closed configuration. As illustrated in FIG. 12, when the adhesive region 170 contacts the glove 10, as the movable lid 130 is moved from a closed configuration to an open configuration, the adhesive force can assist to pull the glove 10 from the enclosed space. The adhesive region 170 can be formed from any suitable adhesive material 170 that may permanently adhere to the inner surface 172 of the rigid lid 120 while releasably adhering to the gloves 10 so that the gloves 10 can then be released from the adhesive region 170 without damaging the integrity of the glove.

The present invention may additionally include a method of loading a dispenser with contents to form a dispensing assembly. For example, the dispenser 100 may be loaded with gloves to form a glove dispensing assembly. The method includes steps of providing a pre-formed dispenser body 102 optionally already having a rigid lid 120 coupled thereto, the dispenser body 120 having one open end (e.g., at the first edge 112 or second edge 114). For example, the dispenser body 102 may be the dispenser body 102 described above and illustrated in FIG. 1. The pre-formed dispenser 100 has an opening 116 for dispensing the contents that can be covered by the lid 120. The opening 116 can be sealed or unsealed when the dispenser 102 is pre-formed. Then, the contents of the dispenser 100 are loaded into the open end of the dispenser 100. For example, an interfolded stack of gloves 10 may be inserted into the dispenser 100 through the open end of first side edge 112. In alternative embodiments, gloves may be inserted into the dispenser without a particular folding configuration, or completely unfolded, or each glove may be individually folded and stacked without interfolding the gloves together. The open end of the dispenser 100 can then be closed and sealed along the first side edge 112 after the contents, e.g. interfolded stack of gloves, have been inserted. If not already sealed, the opening 116 is sealed by the lid 130 or other releasable and resealable seal.

In the method of loading a glove dispensing assembly, the elastic strap 140 can be coupled to the dispenser body 102 before or after the contents, e.g., gloves, are loaded into the enclosed space of the dispenser body 102. For example, in some aspects of the method, the elastic strap can be irremovably coupled to the dispenser body 102 prior to loading the dispenser body 102 with the gloves. In such a configuration, the elastic strap 140 may be stretched into an elongated state during loading of the dispenser body 102. In other aspects of the method, the elastic strap 140 can be coupled to the dispenser 100 after the gloves are loaded into the dispenser, e.g., by coupling the strap coupling element(s) 144 to the retaining element(s) 150 of the rigid lid 120.

When the strap coupling element(s) 144 are utilized to couple the elastic strap 140 to the dispenser 100, the elastic strap 140 can further be uncoupled from the dispenser 100 and then, if desired, reused at a later time. The present inventors have found that the reusable elastic band 140 can provide a cost-effective, versatile solution to enhancing the collapsibility of the dispenser 100 for improved transport and reducing the volume of the dispenser.

FIGS. 13-14B illustrate an elastic strap 140 having a circular or continuous configuration that is configured to extend around the dispenser body 102. For instance, as shown in FIG. 13, the elastic strap 140 may extend continuously around the dispenser body 102. Moreover, as shown in FIG. 14A, the dispenser body 102 may be inserted within an opening 140a of the continuous elastic strap 140. In other words, as shown in FIG. 13, the elastic strap 140 may be wrapped continuously around the dispenser body 102. Thus, in an embodiment, as shown in FIG. 14B, the elastic strap 140 surrounds the dispenser body 102 and is configured to compress the dispenser body 102 as articles (e.g., gloves) are dispensed from the opening 116. In addition, as shown in FIGS. 13 and 14A, the elastic strap 140 may have a slit 140b or other opening formed therein. In such embodiments, the slit 140b can be configured to align with the opening 116 of the dispenser body 102. In this arrangement, contents of the dispenser 100 (e.g., gloves) may be dispensed through the opening 116 and through the slit 140b. Accordingly, in an embodiment, the elastic strap 140 maintains the dispenser 100 tight and compact even as articles are dispensed from the opening 116. In addition, in an embodiment, the elastic strap 140 can replace conventional plastic and/or rigid lids.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. A collapsible dispenser, the dispenser comprising:
a dispenser body including a top, a bottom, and at least one side defining an enclosed space having a volume, wherein the dispenser body is formed from a flexible material;
an opening into the enclosed space being located on the top, the bottom, or a side of the at least one side of the dispenser body; and
an elastic strap coupled to the dispenser body;
wherein the dispenser is configured to collapse as contents contained within the dispenser are dispensed.

2. The dispenser of claim 1, further comprising a rigid lid, wherein the rigid lid comprises a rigid perimeter coupled to the top, bottom, or side of the at least one side of the dispenser body and a movable lid coupled to the rigid perimeter,
the rigid perimeter comprising at least one retaining element configured to retain the elastic strap, and the elastic strap being coupled to the rigid perimeter.

3. The dispenser of claim 2, wherein the at least one retaining element comprises a tab extending from an edge of the rigid perimeter.

4. The dispenser of claim 2, wherein the at least one retaining element comprises a pair of retaining elements disposed on opposing first and second sides of the rigid perimeter.

5. The dispenser of claim 2, wherein the at least one retaining element further comprises at least one indentation in a surface of the rigid perimeter upon which the elastic strap is configured to rest.

6. The dispenser of claim 2, wherein the at least one retaining element comprises at least one slot extending through the rigid perimeter, each slot being formed between an external edge of the rigid perimeter and an internal edge of the rigid perimeter, the at least one slot being configured to receive an elastic band or an external strap or hook.

7. The dispenser of claim 1, wherein the elastic strap comprises an elongated body extending from a first end to a second end, further comprising a strap coupling element at each of the first end and the second end.

8. The dispenser of claim 1, wherein the elastic strap comprises an elongated position and a relaxed position, further wherein the elastic strap is coupled to the dispenser body in the elongated position and, as contents of the dispenser are dispensed, the elastic strap is configured to move into the relaxed position,
wherein as contents of the dispenser are dispensed and the elastic strap moves into the relaxed position, the dispenser body is configured to shrink in size and the volume of the enclosed space is configured to be reduced as the contents of the dispenser are dispensed.

9. The dispenser of claim 1, wherein the elastic strap comprises an elastic ring configured to surround the dispenser body, wherein the elastic ring is configured to cover the opening.

10. The dispenser of claim 9, wherein the elastic ring comprises a slit configured to align with the opening of the dispenser body to permit contents of the dispenser to be dispensed through the slit.

11. A glove dispensing assembly comprising:
a collapsible dispenser comprising:
a dispenser body including a top, a bottom, and at least one side defining an enclosed space having a volume, the top, bottom, and at least one side each being formed from a flexible material; and
a resealable opening into the enclosed space located on the top, the bottom, or a side of the at least one side; and
a stack of interfolded gloves, each glove in the stack including a finger end and a cuff end, the stack including an initial glove and a plurality of subsequent gloves;
the stack of interfolded gloves being disposed in the enclosed space of the dispenser;
the dispenser being configured to collapse as gloves of the stack of gloves contained within the enclosed space are dispensed such that the volume of the enclosed space decreases.

12. The assembly of claim 11, the dispenser further comprising a lid sealing the resealable opening.

13. The dispenser of claim 12, wherein an inner surface of the lid further comprises an adhesive region.

14. The assembly of claim 12, the lid comprising single piece of rigid material comprising an outer perimeter coupled to the flexible material and a movable member configured to move between an open configuration in which the opening is exposed and a closed configuration in which the opening is covered, a movable portion of the lid being a cut-out portion of the single piece of rigid material.

15. The assembly of claim 14, wherein at least a portion of the flexible material is exposed within the outer perimeter of the lid when the movable member is in the open configuration.

16. The assembly of claim 14, the movable portion having a cantilever snap and the outer perimeter having a corresponding slot in which the cantilever snap is configured to lock in place in the closed configuration.

17. The assembly of claim 11 further comprising an elastic strap coupled to the dispenser body; the elastic strap being configured to compress the collapsible dispenser.

18. The assembly of claim 11, further comprising a mounting element coupled to the dispenser body, wherein the mounting element is configured to mount the dispenser to a surface.

19. The assembly of claim 18, wherein the mounting element comprises an adhesive region comprising an adhesive material configured to adhere to the surface.

20. The assembly of claim 18, wherein the dispenser comprises an elongated section comprising the mounting element, and the mounting element comprises one or more openings configured to receive a belt or other accessory therethrough.
